(19) United States
(12) Patent Application Publication
(10) Pub. No.: US 2017/0049724 A1
(43) Pub. Date: Feb. 23, 2017

(54) EXTENDED-RELEASE DOSAGE FORM

(71) Applicant: Mylan Technologies Inc., Morgantown, WV (US)

(72) Inventors: Pavan Bhat, Morgantown, WV (US); Sarat J. Chattaraj, Morgantown, WV (US); Andrew A. Shaw, Morgantown, WV (US)

(21) Appl. No.: 15/218,620

(22) Filed: Jul. 25, 2016

Related U.S. Application Data
(63) Continuation of application No. 12/977,713, filed on Dec. 23, 2010, now abandoned.
Application No. 12/977,713 is a continuation of application No. 11/701,178, filed on Feb. 1, 2007, now abandoned.

(51) Int. Cl. A61K 31/138; A61K 9/50

(52) U.S. Cl. CPC A61K 31/138; A61K 9/5047; A61K 9/5073; A61K 9/5042; A61K 9/5026; A61K 9/5015; A61K 9/501

(57) ABSTRACT

Provided are pharmaceutical formulations comprising sustained release particles each having an inner core bead comprising an active pharmaceutical ingredient an intermediate coating substantially surrounding the inner core bead, and an outer coating substantially surrounding the intermediate coating comprising a pH independent polymer. Also provided is a pharmaceutical formulation comprising two bead populations wherein each of the first and second bead populations have a different drug release profile. Also provided is a method of preparing an extended release dosage composition comprising one or more bead populations.

### CROSS-REFERENCE TO RELATED APPLICATIONS

This application is a continuation of U.S. application Ser. No. 11/701,178, filed on Feb. 1, 2007, the disclosure of which is incorporated herein by reference.

### BACKGROUND OF THE INVENTION

Many sustained release formulations, especially those in tablet and capsule form, are provided with a coating which regulates release of the active ingredient(s) therefrom. Various coating techniques have been utilized to control the rate or the site of the release of the active ingredient in the pharmaceutical formulation.

U.S. Pat. No. 4,587,118 issued to Hsiao discloses a controlled release theophylline oral formulation comprising coated micropellets; each pellet is designed to release theophylline at an approximately constant rate. The pellet comprises a drug-containing core, which is then coated with a mixture of about 70-90% by weight of ethylcellulose and about 10-30% by weight of hydroxypropyl cellulose. The control release characteristics depend on the ratio of ethylcellulose to hydroxypropylcellulose, and the coating thickness.

U.S. Pat. No. 4,957,745 issued to Jonsson et al. describes the art of making a controlled release formulation of a salt of metoprolol comprising a multitude of metoprolol cores prepared by layering the drug onto inert silicon dioxide beads, wherein the core is coated with a metoprolol permeable membrane of essentially ethylcellulose or a mixture of hydroxypropyl methylcellulose and ethylcellulose, the ratio of ethylcellulose to hydroxypropyl methylcellulose depending upon the desired control release characteristics.

U.S. Pat. No. 5,133,974 issued to Paradissis et al. discloses a controlled release formulation comprising a mixture of approximately 0-50% immediate release particles containing a drug, an inert substrate, a binder coated with talc, and up to 100% of extended release particles comprising the immediate release particles coated with a dissolution modifying system containing plasticizers and a film forming agent. Optionally, a drug is included in the coating. Film forming agents utilized therein include ethylcellulose, hydroxypropyl cellulose, hydroxypropyl methylcellulose and mixtures thereof.

U.S. Pat. No. 5,472,708 issued to Chen discloses the art of making a tablet which rapidly disintegrates, comprising a plurality of pellets embedded in the tablet comprising drug containing cores and a swelling agent having a dissolution rate-controlling polymer membrane of a mixture of water-insoluble ethylcellulose and a water soluble film forming polymer, and a permeability reducing agent. The water-soluble polymer is selected from a group containing cellulose acetate phthalate, hydroxypropyl methylcellulose, and polyvinylpyrrolidone. The swelling agent has the property of increasing in volume on exposure to the aqueous environment of use, thus causing rapid release of the drug following bursting of the bead.

U.S. Publication No. 2004/0126427 A1 discloses a unit dosage form for delivering drugs in a sustained release fashion via a system comprising two populations of propranolol-containing particles. Such a drug delivery system is designed by combining immediate release beads and sustained release beads. The sustained release beads are obtained by membrane coating immediate release beads with a water-insoluble polymer such as ethylcellulose or a mixture of a water insoluble polymer and a water soluble polymer such as hydroxylpropylcellulose at a ratio of from about 65:35 to 95:5.

### SUMMARY OF THE INVENTION

In accordance with the present invention, a pharmaceutical formulation has been discovered comprising sustained release particles each having an inner core bead comprising an active pharmaceutical ingredient, an intermediate coating substantially surrounding the inner core bead, and an outer coating substantially surrounding the intermediate coating comprising a pH independent polymer. In accordance with one embodiment of the present invention, the active pharmaceutical ingredient is a water soluble drug. In accordance with another embodiment of the present invention, the water soluble drug is propranolol or a pharmaceutically acceptable salt thereof. In accordance with another embodiment of the present invention, the inner core bead further comprises at least one additive. In accordance with another embodiment of the present invention, the inner core bead further comprises microcrystalline cellulose and hydroxypropyl cellulose. In accordance with another embodiment of the present invention, an amount of the active pharmaceutical ingredient (“API”) in the inner core beads ranges from about 5% to about 80% by weight of the inner core bead. In accordance with another embodiment of the present invention, the amount of the active pharmaceutical ingredient in the inner core beads ranges from about 40% to about 70% by weight of the inner core bead.

In accordance with another embodiment of the present invention, the intermediate coating comprises a component selected from the group consisting of a water soluble component, a water insoluble component, and a mixture of a water soluble component and a water insoluble component. In accordance with another embodiment of the present invention, the water soluble component is selected from the group consisting of hydroxypropyl methylcellulose, lactose, hydroxypropyl cellulose, methylcellulose, polyethylene glycol, polyvinylpyrrolidone, glycerine, salts, propylene glycol, sugar, sugar alcohols, polyvinyl alcohol, and mixtures thereof. In accordance with another embodiment of the present invention, the water insoluble component is selected from the group consisting of ethylcellulose, cellulose acetate butyrate, cellulose acetate, cellulose nitrate, polyvinyl acetate, or mixtures thereof. In accordance with another embodiment of the present invention, the ratio of the water insoluble component to the water soluble component ranges from about 1:6 to about 9:1. In accordance with another embodiment of the present invention, the intermediate coating further comprises at least one additive. In accordance with another embodiment of the present invention, the amount of the intermediate coating applied to the inner core beads ranges from about 0.5% to about 25% by weight of the sustained release particles. In accordance with another embodiment of the present invention, the amount of the intermediate coating applied to the inner core beads ranges from about 1.0% to about 4% by weight of said the sustained release particles.

In accordance with another embodiment of the present invention, the pH independent polymer is selected from the group consisting of a methacrylate based polymer, an acrylate based polymer, a copolymer of acrylate and methacrylate, an acrylate/methacrylate copolymer having quaternary ammonium groups, and an ammonio acrylate/methacrylate copolymer. In accordance with another embodiment of the present invention, the amount of the pH independent polymer ranges from about 40% to about 80% by weight of the outer coating. In accordance with another embodiment of the present invention, the amount of the pH independent polymer ranges from about 50% to about 70% by weight of the outer coating. In accordance with yet another embodiment, the amount of outer coating surrounding the intermediate coated bead population ranges from about 2% to about 35% by weight of the bead or sustained release particle. In accordance with another embodiment of the present invention, the outer coating further comprises at least one additive. In accordance with another embodiment of the present invention, the outer coating further comprises a plasticizer.

In accordance with another embodiment of the present invention, the pharmaceutical formulation further comprises an additional coating. In accordance with another embodiment of the present invention, the additional coating is a sub-coating between the inner core bead and the intermediate coating. In accordance with another embodiment of the present invention, the sub-coating is selected from the group consisting of hydroxypropyl methylcellulose and hydroxypropyl cellulose.

In accordance with another embodiment of the present invention, the sustained release particles are contained within a capsule. In accordance with another embodiment of the present invention, the sustained release particles are compressed into a tablet.

In accordance with another embodiment of the present invention, the ratio of an area under the curve for fasted conditions to an area under the curve for fed conditions ranges from about 0.8 to about 1.25. In accordance with another embodiment of the present invention, the ratio of a peak concentration for fasted conditions to a peak concentration for fed conditions ranges from about 0.8 to about 1.25.

In accordance with another embodiment of the present invention, the pharmaceutical formulations provide a dissolution profile in aqueous media such that about 0.25% to about 14% of the active pharmaceutical ingredient is released after about 1.5 hours; about 5% to about 35% of the active pharmaceutical ingredient is released after about 4 hours; about 20% to about 65% of the active pharmaceutical ingredient is released after about 8 hours; about 50% to about 85% of the active pharmaceutical ingredient is released after about 14 hours; and about 75% to about 100% of the active pharmaceutical ingredient is released after about 24 hours.

In accordance with the present invention, a pharmaceutical formulation has been discovered comprising a first bead population and a second bead population, wherein each of the first and second bead populations contain an inner core bead comprising an active pharmaceutical ingredient, an intermediate coating substantially surrounding the inner core bead, and an outer coating substantially surrounding the intermediate coating comprising a pH independent polymer, and wherein each of the first and second bead populations have different drug release profiles. Such different drug release profiles are developed by selectively altering the amount and/or type of intermediate coatings utilized on the different bead populations, and/or the amount and/or type of outer coatings utilized on the different bead populations, and/or the amount of active pharmaceutical ingredient utilized in the inner cores of the different bead populations. In this manner, a limitless variety of differing drug release profiles can be produced.

In accordance with one embodiment of the present invention, the ratio of the first bead population to the second bead population ranges from about 100:1 to about 1:100.

In accordance with another embodiment of the present invention, the intermediate coating comprises a polymer selected from the group consisting of a water soluble component, a water insoluble component, and a mixture of a water soluble component and water insoluble component. When the intermediate coating is comprised of a mixture of a water soluble component and water insoluble component, the ratio of water insoluble component to water soluble component ranges from about 1:6 to about 9:1.

In accordance with another embodiment of the present invention, the pH independent polymer is selected from the group consisting of a methacrylate based polymer, an acrylate based polymer, an acrylate/methacrylate copolymer, and an ammonio acrylate/methacrylate copolymer.

In accordance with another embodiment of the present invention, the outer coating also contains a plasticizer.

In accordance with another embodiment, additional coatings other than the intermediate and outer coatings are applied. In some embodiments, a sub-coating is applied between the inner core bead and the intermediate coating.

In accordance with another embodiment, the extended release composition further comprises a pharmaceutically acceptable additive.

In accordance with the present invention, a method has been discovered for preparing a pharmaceutical formulation comprising the steps of: preparing inner core beads comprising an active pharmaceutical ingredient, and preparing sustained release particles by sequentially applying: an intermediate coating to the inner core beads such that the intermediate coating substantially surrounds the inner core beads, and an outer coating to the intermediate coated inner core beads comprising a pH independent polymer.

In accordance with the present invention, a method has also been discovered for preparing a pharmaceutical formulation comprising a first bead population and a second bead population wherein the method comprises the steps of preparing inner core beads comprising a water soluble drug, preparing the first and second bead populations by sequentially applying an intermediate coating to the inner core beads such that the intermediate coating substantially surrounds the inner core beads, and an outer coating to the intermediate coated inner core beads comprising a pH independent polymer. The dosage composition is filled with the first and second bead population beads in a ratio ranging from about 100:1 to about 1:100.

Applicants have found that the unique multi-layer coating employed on the inner core beads of the present invention eliminates dose dumping under fed conditions as compared to fasting conditions. Applicants have also found that the pharmaceutical formulation of the present invention eliminates the food effect, i.e. the absorption of the active pharmaceutical ingredient takes place in a reproducible way either in the presence or in the absence of food.

Applicants have also found that pharmaceutical dosage forms containing a single or dual bead population provide pharmacokinetic parameters similar to or better than other extended release dosage forms containing the same active ingredient.

### DETAILED DESCRIPTION

One embodiment of the present invention is a pharmaceutical formulation comprising sustained release particles or beads each having an inner core bead comprising an active pharmaceutical ingredient, an intermediate coating substantially surrounding the inner core bead, and an outer coating substantially surrounding the intermediate coating comprising a pH independent polymer.

While the term “bead” or “sustained release particle” (used interchangeably herein) is used to describe the particulate dosage forms of the present invention, other particulate forms of various sizes and shapes, including pellets, spheroids, spheres, mini-tablets and granules, may be utilized as part of the invention.

Each bead or sustained release particle of the present invention is comprised of an inner core bead onto which at least two subsequent coatings are successively applied. A number of different inner core beads each having different drug release profiles may be obtained by varying the components and/or the amounts of the components in each of the inner core beads.

The inner core bead itself comprises an active pharmaceutical ingredient (“API”) or drug (used interchangeably herein). The active pharmaceutical ingredients may be selected from the groups consisting of antacids, anti-inflammatory substances, coronary dilators, cerebral dilators, peripheral vasodilators, anti-infectives, psychotropics, anti-maniics, stimulants, anti-histamines, decongestants, gastro-intestinal sedatives, anti-anginal drugs, vasodilators, anti-arrhythmics, anti-hypertensive drugs, vasoconstrictors, migraine treatments, anti-coagulants and anti-thrombotic drugs, analgesics, anti-pyretics, hypnotics, sedatives, anti-emetics, anti-nauseants, anti-convulsants, neuromuscular drugs, hyper- and hypoglycemic agents, thyroid and anti-thyroid preparations, diuretics, anti-spasmodics, uterine relaxants, anti-obesity drugs, anabolic drugs, erythropoietic drugs, anti-asthmatics, bronchodilators, expectorants, cough suppressants, mucolytics anti-uricemic drugs and the like.

In some embodiments, the active pharmaceutical ingredient is water soluble, having a solubility greater than 1 part solute to 30 parts solvent. Water soluble API's include salts formed with inorganic and organic acids that are positively charged due to non-covalently attached protons, permanently positively (or negatively) charged molecules, and negatively charged molecules that are salts of weak and strong acids. In other embodiments, the API is freely soluble, having a solubility of about 1 part solute to about 10 parts solvent. In yet other embodiments, the API is soluble, having a solubility of about 1 part solute to about 1 part solvent or less.

Specific APIs may be selected from the group consisting of propranolol, metoprolol tartrate, metoprolol succinate, galantamine, bupropion, diltiazem, oxybutynin, hydrochlorothiazide, metformin, dopamine, ciprofloxacin, vancomycin, norvancomycin, daunorubicin, vinca alkaloids (e.g., vinorelbine), cetrizine, venlafaxine, opioid analgesics (e.g., morphine), tramadol, diltiazem, timolol, trospium, pramipexole, methylphenidate, cimetidine, amphetamine, methamphetamine, cephalexin and pharmaceutically acceptable salts, hydrates, or solvates thereof.

In some embodiments of the present invention, the inner core beads comprise one active pharmaceutical ingredient. In other embodiments of the present invention, the inner core beads comprise a mixture of two or more APIs. In preferred embodiments, the inner core beads comprise an API selected from a water soluble drug. In a most preferred embodiment, the inner core beads comprise propranolol or a pharmaceutically acceptable salt or hydrate thereof.

The amount of active pharmaceutical ingredient contained in the inner core beads will vary depending on the API or APIs contained therein. The amount of API present in the inner core beads ranges from about 5% to about 80% by weight of the inner core bead, preferably ranging from about 40% to about 70% by weight of the inner core bead, and most preferably ranging from about 55% to about 65% by weight of the inner core bead.

In some embodiments, the inner core beads may contain one or more additives selected from the group consisting of binders, fillers, osmotic agents, diluents, absorbents, colorants, dyes, pigments, disintegrants, dispersants, encapsulants, flow aids, hardeners, permeation enhancers, demulcents, stabilizers, disintegrants, tableting aids, glidants, lubricants, plasticizers and wetting agents. Any additive utilized must be pharmaceutically acceptable and compatible with the APT(s) and/or other additive(s). Moreover, any combination of additives may be utilized in the inner core beads of the present invention. The amount of additives in the inner core beads may range from about 1% to about 60% by weight of the inner core bead.

In some embodiments of the present invention, the inner core beads comprise an API and at least one additive. In other embodiments, the inner core beads comprise an API, a binder, and/or a filler, so as to promote adhesion of the API in the bead.

As used herein, the term “binder” means a pharmaceutically acceptable inactive ingredient that holds together or gives strength to a formulation. The binder utilized as part of the inner core beads may be any type of binder suitable for use in the pharmaceutical arts including, but not limited to, polyvinyl-pyrrolidine, hydroxypropyl cellulose, methylcellulose, hydroxypropyl methylcellulose, sugars (e.g., glucose), acacia, carboxymethylcellulose sodium, dextrin, ethylcellulose, gelatin, pregelatinized starch, sodium alginate, zein, and the like or mixtures thereof.

The filler utilized as part of the inner core beads may be any type suitable for use in the pharmaceutical arts including, but not limited to, carboxymethylcellulose, sucrose, mannitol, dextrose, lactose, microcrystalline cellulose, fructose, xylitol, sorbitol, starches, and the like or mixtures thereof.

In some preferred embodiments, the inner core beads comprise an API, microcrystalline cellulose (available under the trade name Avicel® PH 101), and hydroxypropyl cellulose (available under the trade name Klucel® EF). In other preferred embodiments, the inner core beads comprise a water soluble drug, microcrystalline cellulose (available under the trade name Avicel® PH 101), and hydroxypropyl cellulose (available under the trade name Klucel® EF). In yet other preferred embodiments, the inner core beads comprise propranolol or a pharmaceutically acceptable salt thereof, microcrystalline cellulose (available under the trade name Avicel® PH 101), and hydroxypropyl cellulose (available under the trade name Klucel® EF).

The inner core beads are coated with an intermediate coating substantially surrounding the inner core beads. As used herein, “substantially surrounding” means that any coating employed covers from about 40% to about 100% of the inner core bead or the coated inner core bead. The intermediate coating comprises a component selected from the group consisting of a water insoluble component, a water soluble pore forming agent or channeling agent (hereinafter referred to as “water soluble component”), and a mixture of a water insoluble component and a water soluble pore forming agent or channeling agent.

Water insoluble components well known in the art may be utilized in the present invention. The water insoluble component is a pharmaceutically acceptable, non-toxic polymer which is substantially insoluble in aqueous media. Such water insoluble polymers are selected from the group consisting of ethylcellulose, cellulose acetate butyrate, cellulose acetate, cellulose nitrate, polyvinyl acetate, or mixtures thereof.

Water soluble pore forming agents or channeling agents well known in the art may be utilized in the present invention. The water soluble components are pharmaceutically acceptable, non-toxic ingredients which are soluble in water. Such water soluble pore-forming agents or channeling agents are selected from the group consisting of hydroxypropyl methylcellulose, lactose, hydroxypropyl cellulose, methylcellulose, polyethylene glycol, polyvinylpyrrolidone, glycerine, salts, propylene glycol, sugar, sugar alcohols, polyvinyl alcohol or mixtures thereof.

In preferred embodiments, the intermediate coating comprises a mixture of a water insoluble component and a water soluble component. Any combination of water soluble component and water insoluble component may be selected, provided the mixture meets the criteria of the present invention. It is critical that the water soluble component be substantially soluble in the intermediate coating mixture. When the intermediate coating comprising such a mixture is subjected to an aqueous environment, the water soluble component will at least partially dissolve, allowing pores to form in the intermediate coating. It is through these pores that the active pharmaceutical ingredient is released. Thus, when the aqueous medium of the gastrointestinal tract comes into contact with the inner core bead, the water soluble drug starts to dissolve and is released through the pores of the coating, allowing controlled drug release.

In a preferred embodiment, the water insoluble component is ethyl cellulose (available under the trade name Ethocel® Standard 45 Premium) and the water soluble pore forming agent or channeling agent is hydroxypropyl methylcellulose (available under the trade name Pharmacoat® 606).

The ratio of the weight of the water insoluble component to the weight of water soluble component present in the intermediate coating ranges from about 1:6 to about 9:1, preferably ranging from about 1:3 to about 3:1, most preferably ranging from about 1:2 to about 2:1. As the ratio of the water insoluble component to water soluble component is varied, different drug release profiles will be realized.

In some embodiments, the intermediate coating may contain one or more additives including binders, fillers, osmotic agents, diluents, absorbents, colorants, dyes, pigments, disintegrants, dispersants, encapsulants, flow aids, hardeners, permeation enhancers, demulcents, stabilizers, disintegrants, tableting aids, glidants, lubricants, plasticizers, and wetting agents.

By varying the types and/or amounts of components and/or additives utilized in the intermediate coating, different coated inner core beads may be obtained, each having different drug release profiles.

The amount of intermediate coating applied to the inner core beads ranges from about 0.5% to about 25% by weight of the bead or sustained release particle, preferably from about 0.6% to about 15% by weight of the bead or sustained release particle, most preferably from about 1.0% to about 4% by weight of the bead or sustained release particle. By varying the amount of intermediate coating applied to an inner core bead, different beads with different release profiles may be obtained.

An outer coating, substantially surrounding the intermediate coating, is applied to the intermediate coated inner core beads. The outer coating comprises a pH independent polymer. As used herein, the term “pH independent” means that the water permeability of the polymer, and hence its ability to release pharmaceutical ingredients, is not a function of pH and/or is only very slightly dependent on pH. Accordingly, the outer coatings of the present invention are capable of releasing a water soluble drug at a controlled rate which is independent of physiological factors, such as pH in the gastrointestinal tract, which can vary from one subject to another and can vary from time to time for a particular patient, and vary depending on the administration of the dosage form (with or without food).

pH independent polymers well known in the art may be utilized as part of the present invention. In some embodiments, the pH independent polymer may be selected from the group consisting of methacrylate based polymers. In other embodiments, the pH independent polymer may be selected from the group consisting of acrylate based polymers. In yet other embodiments, the pH independent polymer may be selected from the group consisting of copolymers such as acrylate, methacrylate, acrylate/methacrylate, ammonio acrylate, ammonio methacrylate, or ammonio acrylate/methacrylate copolymers. Mixtures of any of the aforementioned classes of polymers or copolymers may be utilized to form the pH independent polymer utilized in the outer coating of the present invention.

In a preferred embodiment, the pH independent coating is an ammonio acrylate/methacrylate copolymer selected from the group consisting of Eudragit® RSPO, Eudragit® RLPO, Eudragit® RL30D, Eudragit® RL100, Eudragit® RS30D, Eudragit® RS100, or Eudragit® RD100, all of which are available from Rohm GmbH.

The amount of pH independent polymer in the outer coating of a bead ranges from about 40% to about 80% by weight of the outer coating, preferably from about 50% to about 70% by weight of the outer coating.

The outer coating may also contain one or more additives including binders, fillers, diluents, absorbents, colorants, dyes, pigments, disintegrants, dispersants, encapsulants, flow aids, hardeners, permeation enhancers, demulcents, stabilizers, disintegrants, tableting aids, anti-tack agents, glidants, lubricants, and wetting agents.

In some embodiments, the outer coating contains an anti-tack agent. As used herein, the term “anti-tack agent” refers to a compound which reduces the adhesiveness or stickiness in a formulation. Representative examples of anti-tack agents include magnesium stearate, calcium stearate, Syloid, colloidal silicon dioxide, or talc.

The amount of anti-tack agent present in the outer coatings ranges from about 5% to about 50% by weight of said outer coating, preferably from about 20% to about 35% by weight of said outer coating.

In other embodiments, the outer coating also contains a plasticizer. As used herein, the term “plasticizer” refers to any compound able to decrease the glass transition temperature and the melt viscosity of a polymer. Representative examples of plasticizers include triacetin, tributyl citrate, triethyl citrate, acetyl tri-n-butyl citrate, diethyl phthalate, castor oil, dibutyl sebacate, acetylated monoglycerides, and mixtures thereof. It will be understood that the plasticizer used may depend on the type of pH independent polymer used in the outer coating composition and the desired drug release profile. The amount of plasticizer used in the outer coating ranges from about 4% to about 40% by weight of said outer coating, preferably from about 8% to about 20% by weight of the outer coating.

The amount of outer coating applied to the intermediate coated inner core beads ranges from about 2% to about 35% by weight of the total bead, preferably from about 4% to about 25% by weight of the total bead, most preferably from about 5% to about 20% by weight of the total bead. By varying the amount of outer coating applied to an intermediate coated bead, different beads with different release profiles may be obtained.

The beads or sustained release particles of the present invention may also contain additional coatings other than the intermediate and outer coatings. Such additional coatings may be positioned in a number of different ways, including positioned directly over the inner core bead and beneath the intermediate coating, positioned as a layer between the intermediate and outer coatings, or positioned over the outer coating.

In general, it is desirable to prime the surface of the inner core beads before applying any coatings. Thus, the bead or sustained release particle may further comprise a subcoating substantially surrounding the inner core beads and applied prior to the application of the intermediate coating, such that the intermediate coating is applied over the subcoating. The subcoating may be a primer selected from the group consisting of hydroxypropylmethylcellulose, hydroxypropylcellulose, polyvinyl alcohol (“PVA”, aminoalkyl methacrylate copolymers (such as Eudragit® E available from Rohm GmBh), or Opadray® Clear, available from Colorcon. In a preferred embodiment, the subcoating comprises Opadry® Clear.

The amount of subcoating applied to the inner core beads ranges from about 1% to about 10% by weight of the total bead, preferably from about 2% to about 6% by weight of the total bead.

The coated beads or sustained release particles of the present invention have a particle size ranging from about 200 μm to about 1700 μm, preferably ranging from about 600 μm to about 1400 μm.

In some embodiments, the pharmaceutical composition is administered as a multi-particulate dosage form, i.e. a dosage form containing a single type of bead or sustained release particle population, i.e. all of the beads or sustained release particles in the formulation have the same constituent components and amounts of components and/or coatings. Of course, different pharmaceutical formulations of varying strengths may be obtained by combining more or less of the beads or sustained release particles in the formulation. The beads or sustained release particles themselves may be encapsulated within gelatin or cellulose-based vegetable capsules or may be compressed into tablets.

In other embodiments, the pharmaceutical formulation comprises a multi-particulate dosage form comprising more than one bead population. For example, such a dosage form could include a first bead population and a second bead population, wherein each of the first and second bead populations comprise an inner core bead comprising an active pharmaceutical ingredient, an intermediate coating substantially surrounding the inner core bead, and an outer coating substantially surrounding the intermediate coating comprising a pH independent polymer, wherein each of the first and second bead populations have different drug release profiles. Of course, such a multi-particulate dosage form is not limited to a formulation comprised only of two bead populations.

Should a two bead population formulation be utilized, the first and second bead population beads contain the components and coatings previously described. The first and second bead population beads differ in: a) the amount and/or type of intermediate coating applied, b) the amount and/or type of outer coating applied, c) the presence or absence of additional coatings or layers, and/or d) the amount of one or more APIs and/or additives contained in the inner core bead. There is no requirement that any of the coatings be the same or that the same inner core beads be utilized in both the first and second bead populations.

By combining different bead populations in any single pharmaceutical dosage form, different drug release profiles can be obtained. Because each of the bead populations may have different drug release profiles, by varying the amounts of each of the bead populations within the dosage form (or even the number of different bead populations present), different formulations of different strengths can be obtained. As an example of a dosage form comprising two bead populations, if it is determined that a first bead population to second bead population ratio of 2:1 provides an efficacious drug release profile (for a particular active pharmaceutical ingredient), different dosage strengths may be realized simply by adding beads to the dosage form, provided that the ratio of first bead population beads to second bead population beads remains the same.

In pharmaceutical dosage forms containing two bead populations, generally the dosage form will contain first bead population beads and second bead population beads in a ratio ranging from about 100:1 to about 1:100. The first and second bead populations may be encapsulated within gelatin or cellulose-based vegetable capsules or compressed into tablets.

Regardless of whether a single bead population or multiple bead populations are utilized in the pharmaceutical dosage form, the capsules and/or tablets containing the beads may each contain one or more additives to further enhance drug release, aid in the tableting or encapsulation processes, or to increase the bulk of the pharmaceutical composition. Representative additives include binders, fillers, diluents, absorbents, colorants, dyes, pigments, desiccants, disintegrants, dispersants, encapsulants, flavor enhancers, flow aids, hardeners, permeation enhancers, demulcents, stabilizers, disintegrants, tableting aids, glidants, lubricants, plasticizers, and wetting agents. It is within the purview of one of ordinary skill in the art to determine how much additive is to be included and the objective that one wishes to accomplish by adding the same. Other pharmaceutically acceptable ingredients selected from the groups consisting of coloring agents, preservatives, artificial sweeteners, flavorants, anti-oxidants, and the like may also be included.

Studies were performed to determine the effect of food on the dosing of the pharmaceutical formulations of the present invention. Area under the curve (“AUCL”) was measured for both fed and fasted conditions. AUCL refers to the area under the total API plasma concentration-time curve from time zero to the last quantifiable concentration. The ratio of an area under the curve for fasted conditions to an area under the curve for fed conditions for the pharmaceutical formulations of the present invention ranges from about 0.8 to about 1.25. Peak concentration (CPEAK) was also measured for both fed and fasted conditions. CPEAK refers to the maximum drug concentration obtained directly from the data without interpolation. The ratio of a peak concentration for fasted conditions to a peak concentration for fed conditions for the pharmaceutical formulations of the present invention ranges from about 0.8 to about 1.25.

Also disclosed are methods of preparing the beads or sustained release particles and the pharmaceutical formulations of the present invention. First, the inner core beads must be prepared. Generally, the inner core beads may be prepared by any process known in the art for producing beads, pellets, spheroids, granules, mini-tablets or particles containing active pharmaceutical ingredients. In some embodiments, the inner core beads may be made by coating an inert particle or a crystal with a drug-containing film-forming formulation. In preferred embodiments, however, the inner core beads are made via a wet granulation process, followed by extrusion and spheronization. Regardless of the method by which the inner core beads are manufactured, such beads are used in the further processing steps.

The individual beads or sustained release particles are prepared by sequentially applying: i) an intermediate coating to the inner core beads such that the intermediate coating substantially surrounds the inner core beads, and ii) an outer coating to the intermediate coated inner core beads comprised of a pH independent polymer. When the pharmaceutical dosage form includes two bead populations, each of the first and second bead populations may be manufactured by the same or different manufacturing processes.

The intermediate and outer coating layers are added to the inner core beads by methods known in the art. In some embodiments, the coating compositions may be applied to the inner core beads in a fluidized bed or pan. In other embodiments, the coating compositions may be applied by spraying or painting the coating compositions onto the inner core beads. In yet other embodiments, the coating compositions are applied in a fluid bed bottom spray or top spray coater by having the beads fluidized in an air stream, and an aqueous dispersion of the coating is sprayed thereon. Various conventional coating apparatuses may be employed to facilitate these methods including a centrifugal fluidized bed coating apparatus, a pan coating apparatus, or a fluidized bed coating apparatus. In the processes described herein, it is to be understood that any solvent used in the preparations is removed by techniques known to one of ordinary skill in the art such as by drying or curing. In a preferred embodiment, the coating layers are applied to the inner core beads via a Wurster bottom spray coater. The method for applying the intermediate coating may be the same or different than the method for applying the outer coating. Apparatus which have been used for coating and/or making beads or sustained release particles are described in U.S. Pat. No. 4,895,733 and in U.S. Pat. No. 5,132,142, each of which are incorporated by reference.

The beads or sustained release particles of the present invention, including the inner core beads, may be made by contacting powder particles, adhering them to each other, and compacting the adhered particles by a rolling movement, wherein the degree of densification is controlled by the energy uptake during the rolling movement. Devices and methods for carrying out such processes are disclosed in U.S. Pat. No. 6,354,728 and in U.S. Patent Application No. 2004/0185111 A1, both of which are incorporated by reference.

In some embodiments, isopropyl alcohol is used as a solvent in preparing the intermediate coating and ethyl alcohol is used as a solvent in preparing the outer coating. In other embodiments, ethyl alcohol is used as a solvent in preparing both the intermediate and outer coatings. In contrast to the use of other solvent systems, such as methylene chloride/methanol, the use of isopropyl alcohol and ethyl alcohol provides an environmentally friendly method of developing coating systems.

The method may further include the step of applying an additional coating to the inner core beads other than the intermediate and outer coatings. Such a coating may be applied directly to the inner core beads before any subsequent processing steps, applied between the intermediate and outer coatings, or applied subsequent to the outer coating. In preferred embodiments, a sub-coating, as disclosed herein, is applied to the inner core beads before the application of the intermediate coating. The additional coating(s) may be applied by any method known in the art and as disclosed herein.

The finished beads are each blended with talc or other additives and encapsulated in gelatin or cellulose-based vegetable capsules or compressed into a tablet to form a pharmaceutical dosage form. In embodiments comprised of two bead populations, appropriate amounts of each bead population are combined, and mixed with talc or other additives.

The following examples further illustrate the invention and its unique characteristics. These examples are not intended to limit the invention in any manner.

### Example 1

### Composition of Inner Core Beads

| Components | mg/g | % |
|---|---|---|
| Propranolol Hydrochloride | 600.00 | 60 |
| Microcrystalline Cellulose (Avicel ® PH 101) | 380.00 | 38 |
| Hydroxypropyl Cellulose (Klucel ® EF) | 20.00 | 2 |
| Total, mg | 1000.0 | |

Example 1 is an example of the components comprising the inner core beads of the present invention. In this particular example, an active pharmaceutical ingredient, propranolol hydrochloride, is combined with two additives. Specifically, hydroxypropyl cellulose (Klucel® RF) was added to purified water to produce a granulating solution. Microcrystalline cellulose (Avicel® PH 101) and the active pharmaceutical ingredient (API), propranolol hydrochloride, were mixed and granulated with the solution described above. The granulated material was then extruded using a Twin Dome Granulator. The extrudate was spheronized using a marumerizer and the spheronized beads were discharged. The discharged beads were dried in a fluid bed, screened, and blended using a “V” blender The resulting product is an inner core bead comprised of about 60% API. Of course, inner core beads of different strengths may be made simply by varying the amount of API or additives therein.

### Example 2

### Composition of Beads or Sustained Release Particles (487.2 mg/g)

Propranolol HCl Extended Release Beads
487.2 mg/g

| Components | mg/g | % |
|---|---|---|
| PART-I | | |
| Propranolol HCl Inner Core Beads | 811.919 | |
| PART-II, Sub-coating | | |
| Clear Opadry ® (YS-2-19017) | 40.5959 | 4.1 |
| Purified Water, USP | (500.683) | |
| PART-III, Intermediate Coating | | |
| Ethyl Cellulose (Ethocel ® Standard 45 | 11.0827 | 1.1 |
| Premium) | | |
| Hydroxypropyl Methylcellulose | 5.9676 | 0.6 |
| (Pharmacoat ® 606) | | |
| Isopropyl Alcohol | (323.956) | |
| PART-IV, Outer Coating | | |
| Eudragit ® RSPO | 83.1522 | 8.3 |
| Talc, Micronized (Alphafil ® 500) | 32.6087 | 3.3 |
| Dibutyl Sebacate | 14.6739 | 1.5 |
| Alcohol 190 Proof (Ethyl Alcohol) | (739.131) | |
| Total-PART-I + II + III + IV + V (solid) | 1000.0 | |
| PART-V | | |
| Talc, Micronized | 0.9735 | 0.1 |
| Fed and Fasting AUCL and CPEAK | | |
| Fed AUCL | 2908 ng * hr/mL | |
| Fed CPEAK | 145.6 ng/mL | |
| Fast AUCL | 2817 ng * hr/mL | |
| Fast CPEAK | 145.7 ng/mL | |
| Fed/Fasting Ratio | | |
| AUCL Fed/Fasting | 1.03 | |
| CPEAK Fed/Fasting | 1.00 | |

This is an example of a sustained release particle or bead population comprising propranolol HCl inner core beads. This particular example contains three coatings—a subcoating, an intermediate coating, and an outer coating, each of which are successively applied to inner core beads (e.g. the inner core beads of example 1).

The subcoating is made by mixing Clear Opadry® and purified water to obtain a subcoating solution that is sprayed onto the inner core beads using a Wurster column in a fluid bed apparatus. The subcoated beads were then screened.

The intermediate coating comprises a mixture of a water soluble component and a water insoluble component. In this particular example, the water insoluble component is ethyl cellulose and the water soluble polymer is hydroxypropyl methylcellulose. The intermediate coating comprises about 1.7% of the total weight of the bead. The intermediate coating was made by mixing isopropyl alcohol, the ethyl cellulose (Ethocel®), and Hypomellose. The resulting dispersion was sprayed onto the subcoated beads using a Wurster column in a fluid bed apparatus. The subcoated beads were dried and cooled in the fluid bed, and screened.

The outer coating comprises a pH independent coating, a binder, and a plasticizer. In this particular example, the pH independent polymer is Eudragit RSPO, the anti-tacking agent is talc, and the plasticizer is dibutyl sebacate. The outer coating comprises about 13.1% of the total weight of the bead. The outer coating was made by mixing the pH independent polymer (Eudragit RSPO®) with ethyl alcohol, and the plasticizer (dibutyl sebacate). The anti-tacking agent (talc) was screened and mixed into this solution to produce the outer coating dispersion. The final, outer coat was sprayed onto the intermediate coated beads using a Wurster column in a fluid bed apparatus. The outer coating comprises about 13.1% of the total weight of the bead. The beads were dried and cooled in a fluid bed, screened, and blended. This specific bead population bead contains 487.2 mg/g of propranolol hydrochloride.

Each of the coating compositions are prepared in aqueous solutions or organic solvents, as indicated. The purified water, isopropyl alcohol, and ethyl alcohol were utilized as solvents in the manufacturing process and were evaporated during processing.

Area under the curve and peak concentration data for both fed and fasted conditions is provided for this example. For the fasting study, 24 human subjects received a 160 mg dose following fasting for at least 10-hours prior to dosing. Each subject then fasted for an additional 4-hours after dosing. For the fed study, 24 human subjects received a 160 mg dose 30-minutes after the administration of a high-fat breakfast (2 eggs fried in butter, 2 strips of bacon, 2 slices of toast with butter, 4 ounces of has brown potatoes, and 8 ounces of whole milk).

### Example 3

### Composition of Beads or Sustained Release Particles (484.8 mg/g)

Propranolol HCl Extended Release Beads
484.8 mg/g

| Components | mg/g | % |
|---|---|---|
| PART-I | | |
| Propranolol HCl Inner Core Beads | 807.9584 | |
| PART-II, Sub-coating | | |
| Clear Opadry ® (YS-2-19017) | 40.3979 | 4.0 |
| Purified Water, USP | (498.2410) | |
| PART-III, Intermediate Coating | | |
| Ethyl Cellulose (Ethocel ® Standard 45 | 13.7858 | 1.4 |
| Premium) | | |
| Hydroxypropyl Methylcellulose | 7.4231 | 0.7 |
| (Pharmacoat ® 606) | | |
| Isopropyl Alcohol | (402.9690) | |
| PART-IV, Outer coating | | |
| Eudragit ® RSPO | 83.1522 | 8.3 |
| Talc, Micronized (Alphafil ® 500) | 32.6087 | 3.3 |
| Dibutyl Sebacate | 14.6739 | 1.5 |
| Alcohol 190 Proof (Ethyl Alcohol) | (739.1310) | |
| Total-PART-I + II + III + IV (solid) | 1000.0 | |
| Fed and Fasting AUCL and CPEAK | | |
| Fed AUCL | 2439.4 ng * hr/mL | |
| Fed CPEAK | 127.6 ng/mL | |
| Fast AUCL | 2354.3 ng * hr/mL | |
| Fast CPEAK | 118.7 ng/mL | |
| Fed/Fasting Ratio | | |
| AUCL Fed/Fasting | 1.04 | |
| CPEAK Fed/Fasting | 1.07 | |

This is an example of a bead or sustained release particle population comprising propranolol HCl inner core beads. This particular example contains three coatings—a subcoating, an intermediate coating, and an outer coating.

The intermediate coating comprises a mixture of a water soluble component and a water insoluble component. Specifically, the water insoluble component is ethyl cellulose and the water soluble component is hydroxypropyl methylcellulose. The intermediate coating comprises about 2.1% of the total weight of the bead.

The outer coating comprises a pH independent coating, an anti-tacking agent, and a plasticizer. The outer coating comprises about 13.1% of the total weight of the bead. Specifically, the pH independent polymer is Eudragit RSPO, the anti-tack agent is talc, and the plasticizer is dibutyl sebacate. This specific second bead population bead contains 484.8 mg/g of propranolol hydrochloride and was made according to the methods described in Example 2. Each of the coating compositions are prepared in aqueous solutions or organic solvents, as indicated.

Area under the curve and peak concentration data for both fed and fasted conditions is provided for this example. For the fasting study, 23 human subjects received a 160 mg dose following fasting for at least 10-hours prior to dosing. Each subject then fasted for an additional 4-hours after dosing. For the fed study, 23 human subjects received a 160 mg dose 30-minutes after the administration of a high-fat breakfast (2 eggs fried in butter, 2 strips of bacon, 2 slices of toast with butter, 4 ounces of has brown potatoes, and 8 ounces of whole milk).

### Example 4

### Composition of Beads or Sustained Release Particles (517.82 mg/g)

Propranolol HCl Extended Release Beads
517.82 mg/g

| Components | mg/g | % |
|---|---|---|
| PART-I | | |
| Propranolol HCl Inner Core Beads | 863.03303 | |
| PART-II, Sub-coating | | |
| Clear Opadry ® (YS-2-19017) | 43.1517 | 4.3 |
| Purified Water, USP | (532.204) | |
| PART-III, Intermediate Coating | | |
| Ethyl Cellulose (Ethocel ® Standard 45 | 14.7255 | 1.5 |
| Premium) | | |
| Hydroxypropyl Methylcellulose | 7.92912 | 0.8 |
| (Pharmacoat ® 606) | | |
| Isopropyl Alcohol | (402.9690) | |
| PART-IV, Outer coating | | |
| Eudragit ® RSPO | 44.4101 | 4.4 |
| Talc, Micronized (Alphafil ® 500) | 17.4157 | 1.7 |
| Dibutyl Sebacate | 7.8371 | 0.8 |
| Alcohol 190 Proof (Ethyl Alcohol) | (394.756) | |
| PART-V | | |
| Talc, Micronized | 1.49775 | 0.1 |
| Total-PART-I + II + III + IV + V (solid) | 1000.0 | |
| Fed and Fasting AUCL and CPEAK | | |
| Fed AUCL | 3988.3 ng * hr/mL | |
| Fed CPEAK | 175.7 ng/mL | |
| Fast AUCL | 4041.3 ng * hr/mL | |
| Fast CPEAK | 198.3 ng/mL | |
| Fed/Fasting Ratio | | |
| AUCL Fed/Fasting | 0.99 | |
| CPEAK Fed/Fasting | 0.89 | |

This is an example of a bead or sustained release particle population comprising propranolol HCl inner core beads. This particular example contains three coatings—a subcoating, an intermediate coating, and an outer coating.

The intermediate coating is comprised of a mixture of a water soluble component and a water insoluble component. Specifically, the water insoluble component is ethyl cellulose and the water soluble component is hydroxypropyl methylcellulose. The intermediate coating comprises about 2.3% of the total weight of the bead. The outer coating comprises a pH independent coating, an anti-tacking agent, and a plasticizer.

The outer coating comprises about 6.9% of the total weight of bead. Specifically, the pH independent polymer is Eudragit RSPO, the binder is talc, and the plasticizer is dibutyl sebacate. This specific second bead population bead contains 517.82 mg/g of propranolol hydrochloride and was made according to the methods described in Example 2. Each of the coating compositions are prepared in aqueous solutions or organic solvents, as indicated.

Area under the curve and peak concentration data for both fed and fasted conditions is provided for this example. For the fasting study, 36 human subjects received a 160 mg dose following fasting for at least 10-hours prior to dosing. Each subject then fasted for an additional 4-hours after dosing. For the fed study, 36 human subjects received a 160 mg dose 30-minutes after the administration of a high-fat breakfast (2 eggs fried in butter, 2 strips of bacon, 2 slices of toast with butter, 4 ounces of has brown potatoes, and 8 ounces of whole milk).

### Example 5

### Composition Comprising a First Bead Population and a Second Bead Population (506.3 mg/g)

Mix of Propranolol HCl Extended Release Beads
65% Beads from Example 4 (517.82 mg/g)
35% Beads from Example 3 (484.8 mg/g)

| Components | mg/g | % |
|---|---|---|
| PART-I | | |
| Propranolol HCl Inner Core | 843.7569 | |
| Beads | | |
| PART-II, Sub-coating | | |
| Clear Opadry ® (YS-2-19017) | 42.1879 | 4.3 |
| Purified Water, USP | (520.3170) | |
| PART-III, Intermediate Coating | | |
| Ethyl Cellulose (Ethocel ® | 14.3966 | 1.4% |
| Standard 45 Premium) | | |
| Hydroxypropyl Methylcellulose | 7.7520 | 0.8% |
| (Pharmacoat ® 606) | | |
| Isopropyl Alcohol | (420.8239) | |
| PART-IV, Outer coating | | |
| Eudragit ® RSPO | 57.9698 | 5.8% |
| Talc, Micronized (Alphafil ® | 22.7333 | 2.3% |
| 500) | | |
| Dibutyl Sebacate | 10.2300 | 1.0% |
| Alcohol 190 Proof (Ethyl | (515.2873) | |
| Alcohol) | | |
| PART-V | | |
| Talc, Micronized | 0.9735 | 0.1% |
| Total-PART-I + II + III + IV + V | 1000.0 | |
| (solid) | | |
| Fed and Fasting AUCL and CPEAK | | |
| Fed AUCL | 4052 ng * hr/mL | |
| Fed CPEAK | 154.2 ng/mL | |
| Fast AUCL | 4310 ng * hr/mL | |
| Fast CPEAK | 180.6 ng/mL | |
| Fed/Fasting Ratio | | |
| AUCL Fed/Fasting | 0.94 | |
| CPEAK Fed/Fasting | 0.85 | |

Example 5 provides an illustration of a combination of two different sustained release particle or bead populations mixed to produce a pharmaceutical formulation. This particular example highlights a pharmaceutical dosage form containing 65% of a first bead population (from Example 4) and 35% of a second bead population (from Example 3), whereby a specific drug release profile is provided.

The capsules of Example 5 were manufactured by a blend-encapsulate process. In addition to the two populations of beads, the capsules contained Talc, Micronized (Alphafil 500) (a glidant) Each population of beads was separately added to a V blender and mixed with the talc prior to encapsulation. The actual target capsule fill weight was calculated using an assigned potency factor, which was determined after each bead blending process.

Area under the curve and peak concentration data for both fed and fasted conditions is provided for this example. For the fasting study, 99 human subjects received a 160 mg dose following fasting for at least 10-hours prior to dosing. Each subject then fasted for an additional 4-hours after dosing. For the fed study, 98 human subjects received a 160 mg dose 30-minutes after the administration of a high-fat breakfast (2 eggs fried in butter, 2 strips of bacon, 2 slices of toast with butter, 4 ounces of has brown potatoes, and 8 ounces of whole milk).

### Example 6

The in vitro dissolution of the capsules were evaluated in 0.1 N HCl (pH 1.2, 900 mL) for 1.5 hours and in buffer (pH 6.8, 900 mL) in 40 wire mesh baskets at 100 rpm. The samples were pulled at 1.5 hours, 4.0 hours, 8 hours, 14 hours and 24 hours. The results are presented in the following table.

| | Example 3 | Example 4 | Example 5 |
|---|---|---|---|
| Hours | 484.8 mg/g | 517.82 mg/g | Capsule |
| 1.5 | 2% | 3% | 3% |
| 4 | 7% | 20% | 18% |
| 8 | 24% | 52% | 42% |
| 14 | 55% | 77% | 65% |
| 24 | 83% | 92% | 83% |

In general, the in vitro dissolution is such that about 0.25% to about 14% of the API is released after about 1.5 hours; about 5% to about 35% of the API is released after about 4 hours; about 20% to about 65% of the API is released after about 8 hours; about 50% to about 85% of the API is released after about 14 hours; and about 75% to about 100% of the API is released after about 24 hours.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A pharmaceutical formulation comprising sustained release particles having an inner core bead comprising a granulate of an active pharmaceutical ingredient, an intermediate coating substantially surrounding said inner core bead, and an outer coating comprising a pH independent polymer substantially surrounding said intermediate coating, wherein said formulation provides a dissolution profile in aqueous media such that about 50% to about 85% of said active pharmaceutical ingredient is released after about 14 hours, and about 75% to about 100% of said active pharmaceutical ingredient is released after about 24 hours.

2. The pharmaceutical formulation of claim 1, wherein said active pharmaceutical ingredient is propranolol.

3. (canceled)

4. The pharmaceutical formulation of claim 1, wherein said inner core bead further comprises at least one additive selected from the group consisting of binders, fillers, osmotic agents, diluents, absorbents, colorants, dyes, pigments, disintegrants, dispersants, encapsulants, flow aids, hardeners, permeation enhancers, demulcents, stabilizers, disintegrants, tableting aids, glidants, lubricants, plasticizers, and wetting agents.

5. (canceled)

6. The pharmaceutical formulation of claim 1, wherein said inner core bead further comprises microcrystalline cellulose and hydroxypropyl cellulose.

7. The pharmaceutical formulation of claim 1, wherein an amount of said active pharmaceutical ingredient in said inner core bead ranges from about 5% to about 80% by weight of said inner core bead.

8. (canceled)

9. The pharmaceutical formulation of claim 1, wherein said intermediate coating comprises a component selected from the group consisting of a water soluble component, a water insoluble component, and a mixture of a water soluble component and a water insoluble component.

10. The pharmaceutical formulation of claim 9, wherein said water soluble component is selected from the group consisting of hydroxypropyl methylcellulose, lactose, hydroxypropyl cellulose, methylcellulose, polyethylene glycol, polyvinylpyrrolidone, glycerine, salts, propylene glycol, sugar, sugar alcohols, polyvinyl alcohol, and mixtures thereof.

11. The pharmaceutical formulation of claim 9, wherein said water insoluble component is selected from the group consisting of ethylcellulose, cellulose acetate butyrate, cellulose acetate, cellulose nitrate, polyvinyl acetate, and mixtures thereof.

12-13. (canceled)

14. The pharmaceutical formulation of claim 9, wherein said intermediate coating further comprises at least one additive selected from the group consisting of binders, fillers, osmotic agents, diluents, absorbents, colorants, dyes, pigments, disintegrants, dispersants, encapsulants, flow aids, hardeners, permeation enhancers, demulcents, stabilizers, disintegrants, tableting aids, glidants, lubricants, plasticizers, and wetting agents.

15. (canceled)

16. The pharmaceutical formulation of claim 1, wherein the amount of said intermediate coating applied to said inner core bead ranges from about 0.5% to about 25% by weight of said sustained release particles.

17. (canceled)

18. The pharmaceutical formulation of claim 1, wherein said pH independent polymer is selected from the group consisting of a methacrylate based polymer, an acrylate based polymer, a copolymer of acrylate and methacrylate, an acrylate/methacrylate copolymer having quaternary ammonium groups, and an ammonio acrylate/methacrylate copolymer.

19. The pharmaceutical formulation of claim 18, wherein the amount of said pH independent polymer ranges from about 40% to about 80% by weight of said outer coating.

20. (canceled)

21. The pharmaceutical formulation of claim 1, wherein said outer coating further comprises at least one additive selected from the group consisting of binders, fillers, diluents, absorbents, colorants, dyes, pigments, disintegrants, dispersants, encapsulants, flow aids, hardeners, permeation enhancers, demulcents, stabilizers, disintegrants, tableting aids, glidants, lubricants, plasticizers, and wetting agents.

22. (canceled)

23. The pharmaceutical formulation of claim 1, wherein an amount of said outer coating applied to said intermediate coated beads ranges from about 2% to about 35% by weight of said sustained release particle.

24. (canceled)

25. The pharmaceutical formulation of claim 1, wherein said outer coating further comprises a plasticizer selected from the group consisting of dibutyl sebacate, dibutyl phthalate, diethyl phthalate, triethyl citrate, tributyl citrate, benzyl benzoate, glycerin, propylene glycol, polyethylene glycol, triacetin, acetylated monoglycerides, citrate esters, phthalate esters, and mixtures thereof.

26-29. (canceled)

30. The pharmaceutical formulation of claim 1, further comprising one or more additives selected from the group consisting of binders, fillers, diluents, anti-tack agents, absorbents, colorants, dyes, artificial sweeteners, pigments, dispersants, encapsulants, flavor enhancers, flow aids, anti-oxidants, hardeners, permeation enhancers, demulcents, stabilizers, disintegrants, tableting aids, preservatives, glidants, lubricants, plasticizers, and wetting agents.

31. (canceled)

32. The pharmaceutical formulation of claim 1, wherein a ratio of an area under the curve for fed conditions to an area under the curve for fasted conditions ranges from about 0.8 to about 1.25.

33. The pharmaceutical formulation of claim 1, wherein a ratio of a peak concentration for fed conditions to a peak concentration for fasted conditions ranges from about 0.8 to about 1.25.

34-35. (canceled)

36. A pharmaceutical formulation comprising:
a first bead population, and
a second bead population,
wherein each of said first and second bead populations comprise:
an inner core bead comprising a granulate of an active pharmaceutical ingredient,
an intermediate coating substantially surrounding said inner core bead, and
an outer coating comprising a pH independent polymer substantially surrounding said intermediate coating, and
wherein each of said first and second bead populations have different drug release profiles, said formulation providing a dissolution profile in aqueous media such that about 50% to about 85% of said active pharmaceutical ingredient is released after about 14 hours, and about 75% to about 100% of said active pharmaceutical ingredient is released after about 24 hours.

37-64. (canceled)

65. A pharmaceutical formulation comprising sustained release particles having an inner core bead comprising a granulate of an active pharmaceutical ingredient, an intermediate coating substantially surrounding said inner core bead, and an outer coating comprising of a pH independent polymer substantially surrounding said intermediate coating, said intermediate coating comprising a mixture of a water insoluble component and a water soluble component, wherein the ratio of said water insoluble component to said water soluble component ranges from about 2:1 to about 3:1, said pharmaceutical formulation being insensitive to a food effect and capable of once-a-day dosing.

66-92. (canceled)
